(19) United States
(12) Patent Application Publication
(10) Pub. No.: US 2026/0250741 A1
(43) Pub. Date: Aug. 27, 2026

(54) DEVELOPMENT OF CELL MEDIUM AND FEED ON MAMMALIAN CELLS

(71) Applicant: Evonik Operations GmbH, Essen (DE)

(72) Inventors: Suki ROY, Sydney, New South Wales (AU); Sanjanaa NAGARAJAN, Singapore (SG); Florian BÖHL, Neckargemünd (DE); Lingzhi HUANG, Singapore (SG); Rose WHELAN, Ambrosden Oxfordshire (GB); Kit Yeng WONG, Singapore (SG)

(73) Assignee: Evonik Operations GmbH, Essen (DE)

(21) Appl. No.: 19/160,104

(22) PCT Filed: Feb. 27, 2024
(86) PCT No.: PCT/EP2024/054934

§ 371 Date: Aug. 27, 2025

(30) Foreign Application Priority Data
Mar. 2, 2023 (EP) 23159542.2

(51) Int. Cl. C12Q 1/6809; C12N 5/00; C12N 5/071; C12Q 1/6827

(52) U.S. Cl. CPC C12Q 1/6809; C12N 5/0018; C12N 5/0682; C12Q 1/6827; C12N 2500/05; C12N 2500/32; C12N 2500/34; C12N 2500/38; C12N 2500/60; C12N 2510/04; C12Q 2600/158

(57) ABSTRACT

The present invention is related to a DNA array-based method of assessing the effect of at least one test component of cell medium on at least one phenotype of interest of a test mammalian cell line cultured in cell media comprising the test component, the method comprising the steps of:
(a) determining a test methylation profile of one or more pre-selected methylation sites within the DNA of the test cell line;
(b) comparing the test methylation profile obtained from (a) with at least one control methylation profile from the same strain of mammalian cell line cultured in cell media without the test component; and
   wherein a significant similarity in the test methylation profile of (a) compared to the control methylation profile, is indicative of the test cell having the phenotype of interest and the test component not having an effect on the phenotype of interest; and
wherein a significant difference in the test methylation profile of (a) compared to the control methylation profile, is indicative of the test cell having the phenotype of interest and the test component having an effect on the phenotype of interest and wherein the method comprises a further step of:
(c) comparing the test methylation profile obtained from (a) with
   (i) at least one first reference methylation profile obtained from a first mammalian reference cell line that displays at least one phenotype of interest; and/or
   (ii) at least one second reference methylation profile obtained from a second mammalian reference cell line that does not display the phenotype of interest; and
      wherein the reference cell lines are not in contact with the test component; and
      wherein a significant similarity in the test methylation profile of (a) compared to the first or second reference methylation profile, is indicative of the test cell having the phenotype of interest or not having the phenotype of interest respectively; and
      wherein a difference in the test methylation profile of (a) compared to the first or second reference methylation profile, is indicative of the test cell not having the phenotype of interest or having the phenotype of interest.

### FIELD OF THE INVENTION

The present invention relates to a method based on epigenetics, namely a DNA Methylation based method for quantitatively and qualitatively assessing the effect of cell media/feed or a component thereof on at least one phenotype of interest, for example cell survival, performance and/or target protein production in mammalian cells and cell stability prior, during or after the actual production of the protein. In particular, the measure of differential methylation of promotors and/or CpG sites of mammalian cells in the presence of at least one component of the cell medium using DNA methylation array may provide an insight into the effect of the component on quantitative and qualitative production of the target protein by the mammalian cells.

### BACKGROUND OF THE INVENTION

Mammalian cells are used not only the field of research but also in manufacturing of recombinant proteins, for example therapeutic proteins (e.g. monoclonal antibodies). These mammalian cells are grown and cultured in cell media which generally comprises serum or protein hydrolysate components (i.e., peptones and tryptones). These components contain growth factors and a wide variety of other uncharacterized elements beneficial to cell growth and culture. However, they also contain uncharacterized elements that reduce growth or otherwise negatively impact recombinant protein production. They can also be an unwelcome potential source of variability.

Usually, in most mammalian cell lines, initial protein expression from the cell line is high, however the production reduces during prolonged culture. This results in decreased process yield, impacts timelines and increases costs. Changes in cell culture environment can result in an alteration of cell behaviour and protein productivity of the producer cell line.

The cell culture media provides sufficient nutrients to all cells, for optimal growth, high productivity, and quality. Optimizing an appropriate media is crucial for cell line development, and bioprocessing. Media composition and optimization have a strong effect on cell health, metabolism, protein production and quality. For example, several studies have concluded that media composition widely affects protein quality attributes such as glycosylation pattern, aggregation, and charge variant. Individual media ingredient composition and their relative concentration can widely alter media performance. However, the impact of media optimization is not always uniform, as different cell lines producing various recombinant proteins might respond in a different way to a given medium formulation. Thus, media optimization has been a topic of ongoing research to improve cell growth, protein productivity and quality.

In general, medium optimization efforts involve several rounds of optimization by analysing the used media for utilization of individual components and monitoring the effect of supplementation on the desired outcome of the culture. A media comprises a large number of ingredients and each ingredient will have a large number of possible concentration-dependent combinations, thus making the optimization process burdensome, highly complex, limiting and time-consuming. To reduce the number of physical experiments, mathematical models such as the Design of experiment (DOE) have been developed to predict the outcome of a media formulation, however, these algorithms do need data inputs from the cell culture system. That's why, a media development project, starting from scratch, might take months to optimize a suitable formulation. Chinese Hamster Ovary (CHO) cells are known to be the workhorses for the industrial production of recombinant therapeutic proteins since 1987 and are hence widely used for biologics production. About 70% of all recombinant biopharmaceutical proteins and all monoclonal antibodies approved since 2016 are being manufactured in CHO cells. Several advantages of utilizing CHO for biologics production include tolerance to genetic manipulations, ease of adaptation to manufacturing process scales, rapid growth rates, and ability to perform human-compatible post-translational modifications. However, the biologics production system in CHO faces a bottleneck due to the loss of protein productivity over time.

In the current market of bioproduction, an efficient and robust analytical method is in high demand to monitor media composition to improve the optimization and development of media formulation. Such a method can fulfil the multi-metabolic demands of different types of clones and cell lines. Unfortunately, most of the media that are currently being used in the market are not fully optimized due to a lack of robust analytical tools. Accordingly, there is still a need in the art for such a robust analytical tool to optimise cell media for mammalian cell lines, particularly, CHO cells.

### BRIEF DESCRIPTION OF FIGURES

FIG. 1 is a plot showing the results of Principle Component Analysis (PCA) of 122 differentially methylated regions (DMRs) identified.

FIG. 2 is a plot showing the results of Principle Component Analysis (PCA) of 289 differentially methylated regions (DMRs) identified.

FIG. 3 is a picture of the media adaptation experiment cell culture workflow.

FIG. 4 is a plot showing the results of PCA analysis of Media adaptation experiment with all methylated CpG sites.

FIG. 5 is a plot showing the results of PCA analysis of Media adaptation experiment with differentially methylated CpG sites.

### DESCRIPTION OF THE INVENTION

The present invention attempts to solve the problems above by providing a method using DNA methylation patterns to distinguish the effect of one component of cell media from another on a cell which is cultured in the cell media. This method according to any aspect of the present invention is not only accurate and reliable but it also saves time, costs and effort needed to determine the effect of a particular cell media or component thereof on a phenotype of interest of the cell, for example the cell's general health and/or performance in the short or long term. In particular, the effect of the cell media or component thereof on the stability, growth, ability to produce proteins by the cell cultured in the cell media may be determined and/or predicted for the long run using the method according to any aspect of the present invention, without having to monitor the cell or a group of cells for a long time. In particular, the cell may be a mammalian cell. The method according to any aspect of the present invention provides for methods of predicting the cell's performance based on the DNA methylation profile of a cell. In particular, the method according to any aspect of the present invention also provides methods of monitoring the effect of a type of cell medium or a component thereof or even a regimen on the current performance or future performance of the cell. The method according to any aspect of the present invention further provides a means of managing a cell culturing operation by determining suitable cell mediums and/or components thereof to culture the cell in to achieve the best performance and/or prototype of interest from the cell. Improved management can thereby optimize cell performance and the heterologous proteins produced therefrom.

The present invention is based on the finding that components of cell medium can change the epigenome of the cell through epigenetics. In particular, the capability to adapt to the environment and maintain the adapted biological pattern depends on epigenetic mechanisms, including DNA methylation. More in particular, the present invention is based on the finding that cell medium may also result in changes in epigenetic mechanisms of the cell, including DNA methylation patterns and these patterns may be passed down to the different products that may derive from the cell.

The inventors have unexpectedly found that this property can be utilized to identify “epigenetic fingerprints” on the genome that are specific to a component of cell medium that may improve general cell stability and/or performance of not just one cell being fed the component but possibly all the cells that are grown in the cell medium or components thereof. Based on these findings, the present invention provides means to identify the specific effect short term and in the long run of any component of cell medium on the general cell stability and/or performance of the cell cultured in the medium. In particular, the method according to any aspect of the present invention may be used to determine if a specific component of any cell medium has a positive or negative effect on the general cell stability and/or performance of the cell. For example, a component X in the cell medium may improve general cell stability and/or performance of the cell cultured in the cell medium in the short and/or long run resulting in the cell having relatively good cell stability and/or performance. In another example, a component Y in the cell medium may worsen the existing general cell stability and/or performance of the cell cultured in the cell medium resulting in the cell having relatively bad cell stability (i.e. cell exhaustion and low cell survivability) and/or performance. More in particular, the method according to any aspect of the present invention may be used to determine if a particular component of cell medium or the cell medium in itself has a positive or negative effect on the general cell stability and/or performance of the cell per se. In this way, the method according to any aspect of the present invention may then be used to accurately, reliably and quickly determine the specific effect of a component in cell medium on the cell and based on these results, it can be decided if the component should be included in the cell medium of the cell or should be removed from the cell medium in which the cell is cultured.

According to one aspect of the present invention, there is provided a DNA array-based method of assessing the effect of at least one test component of cell medium on at least one phenotype of interest of a test mammalian cell line cultured in the cell medium comprising the test component, the method comprising the steps of:
(a) determining a test methylation profile of one or more pre-selected methylation sites within the DNA of the test cell line;
(b) comparing the test methylation profile obtained from (a) with at least one control methylation profile from the same strain of mammalian cell line cultured in cell medium without the test component; and
wherein a significant similarity in the test methylation profile of (a) compared to the control methylation profile, is indicative of the test cell having the phenotype of interest and the test component not having an effect on the phenotype of interest; and
wherein a significant difference in the test methylation profile of (a) compared to the control methylation profile, is indicative of the test cell having the phenotype of interest and the test component having an effect on the phenotype of interest.

As used herein, the term ‘phenotype of interest’ in connection with a mammalian cell refers to the cell displaying at least one the following characteristics selected from the group consisting of optimal heterologous protein production, phenotypic homogeneity, protein quality, optimal carbohydrate metabolism, optimal amino acid metabolism, optimal lipid metabolism, optimal cell survivability and combinations thereof. In particular, the phenotype of interest refers to a characteristic that the mammalian cell according to any aspect of the present invention displays that is beneficial to the survival of the cell, suitability of the cell for protein production and the overall protein production of the cell. In particular, ‘phenotype of interest” is not only limited to protein productivity but also able to assess the optimal condition for heterologous protein production, phenotypic homogeneity, protein quality, optimal carbohydrate metabolism, optimal amino acid metabolism, optimal lipid metabolism, and/or optimal cell survivability.

The term ‘suitability’ as used herein, refers to a mammalian cell line that is fit for optimal heterologous protein production. In one example, a mammalian cell line may be considered suitable for optimal heterologous protein production before a transgene is introduced into the cell. In this case, the mammalian cell line may have at least one phenotype of interest or characteristics that enable the cell line to grow well and allow for easy uptake of the transgene of interest and following the uptake of the transgene, allow for optimal heterologous protein production, where the protein is a product of the transgene of interest. These characteristics or phenotype of interest include at least optimal glucose consumption, growth rate, lactic acid production, ammonia accumulation and the like. When a mammalian cell line is confirmed of displaying at least one of these phenotypes of interest, the mammalian cell line may be considered suitable for optimal heterologous protein production when the transgene of interest is introduced into the cell.

In another example, a mammalian cell line may be considered suitable for optimal heterologous protein production after the transgene has been introduced into the cell. In this case, a mammalian cell line is genetically modified using methods known in the art to introduce a transgene into the cell and the genetically modified cell is capable of optimal heterologous protein production where the protein is a product of translation of the transgene. The mammalian cell line in this example, may have a least one phenotype of interest that enables the genetically modified cell line to have good viability and optimal target protein production. These phenotypes of interest may include cell viability (survivability), protein productivity (in terms of protein quantity and quality), phenotypic homogeneity, cell exhaustion, and the like. Accordingly, the method according to any aspect of the present invention may be used on a mammalian cell line that has been genetically modified (i.e. with transgene introduced into the cell line) or on a mammalian cell line that has not yet been genetically modified. In both cases, the mammalian cell lines for use in heterologous protein production.

As used herein, the term ‘transgene’ refers to a gene that is taken from the genome of one organism and inserted into the genome of another organism by artificial techniques used in genetic modification. For example, a human gene is artificially introduced into the genome of mammalian cells for the production of at least one protein of interest, particularly therapeutic proteins.

As used herein, the term ‘therapeutic protein’ refers to genetically engineered versions of naturally occurring human proteins. Examples of therapeutic proteins include antibody-based drugs, anticoagulants, blood factors, bone morphogenetic proteins, engineered protein scaffolds, enzymes, growth factors, hormones, interferons, interleukins and the like.

As used herein, the term ‘cell survivability’ refers to the capability of a cell to be viable and perform cell proliferation. Cell viability is a measure of the proportion of live cells within a population. Cell proliferation refers to an increase in cell number due to cell division. The assays that are commonly used to test cell survivability include BrdU Cell Proliferation Assay, MTT Cell Proliferation Assays, trypan blue cell counting, and ATP Cell Viability Assays.

As used herein, the term ‘cell exhaustion’ refers to the state of the cell where it loses its capability to perform metabolic activity including heterologous protein production. Cell exhaustion can be determined by Metabolite Detection Assays.

As used herein, the term ‘phenotypic homogeneity’ refers to a state when all the cells in a population exhibit the same phenotype under a certain condition.

The term ‘heterologous protein production’ as used herein refers to the production of a protein which is not endogenous to the cell. It means an expression of a gene or part of a gene, particularly a transgene in a host mammalian cell which does not naturally express this gene. The assays that are commonly used to quantify heterologous protein production include enzyme-linked immunosorbent assay (ELISA), chromatography & bioprocess analyser. The term ‘host cell’ as used herein refers to a cellular system for the expression of heterologous protein. For example, CHO cells are the main hosts for the production of various therapeutic proteins.

The term ‘optimal heterologous protein production’ herein refers to mammalian cells that are capable of high-level protein production, particularly during industrial production or large-scale production of recombinant proteins, where the protein is usually a functional protein that is not naturally occurring in the wild-type mammalian cell. In particular, for optimal heterologous protein production a mammalian cell line has minimized metabolic burdens and toxic effects to the cell. More in particular, ‘optimal heterologous protein production’ refers to high level protein production where the mammalian cell line, for example CHO cell not only produces a high yield of the protein of interest but also that the protein production is constantly maintained over the period of production (i.e., the prolonged period of culture) such that the quality of the protein produced is also consistent and maintained. In particular, for a mammalian cell according to any aspect of the present invention to be capable of ‘optimal heterologous protein production’, the cell must at least display one of more of the following phenotypes of interest: phenotypic homogeneity, protein productivity, and protein quality. More in particular, for ‘optimal heterologous protein production’, the mammalian cell may comprise phenotypic homogeneity and protein productivity, or phenotypic homogeneity, and protein quality, or protein productivity, and protein quality, or phenotypic homogeneity, protein productivity, and protein quality.

The term ‘protein productivity’ as used herein refers to a measure of the amount of protein made per viable cell at a single titre point. It is calculated by dividing the titre (mg) by the viable cell density (VCD or cells/ml), and the final measurement is represented as the amount of protein per cell (mg/cell).

The term ‘protein quality’ refers to the posttranslational modification of the protein that determines the efficacy and function of the protein. The modifications generally include phosphorylation, glycosylation, ubiquitination, methylation, acetylation, protein folding etc. For example, protein glycosylation is a critical quality attribute that modulates the efficacy, stability, and half-life of a therapeutic protein. Protein quality can be determined using Immunoprecipitation based techniques, Biochemical Assays, Mass spectrometry (MS) and the like.

The term ‘carbohydrate metabolism’, as used herein refers to almost all or all of the biochemical processes responsible for the metabolic formation, breakdown, and interconversion of carbohydrates in cells. It involves multiple pathways such as glycolysis, gluconeogenesis, glycogenolysis, and glycogenesis. For example, glycolysis is one of the key metabolic pathways of CHO cells. Through glycolysis, CHO cells consume glucose as the main carbon source for energy production and generate lactate as the most common metabolic by-product. Particularly, the term ‘optimal carbohydrate metabolism’ refers to the ideal or best carbohydrate metabolism possible by a CHO cell.

Similarly, the term ‘amino acid metabolism’ as used herein refer to the whole of the biochemical processes responsible for the metabolic formation, breakdown, and interconversion of amino acids in cells. Amino acids are the basic building blocks of proteins and constitute all proteinaceous material of the cell including the cytoskeleton, protein component of enzymes, receptors, and signalling molecules. In addition, amino acids are utilized for the growth and maintenance of cells. For example, glutaminolysis is a key metabolic pathway of CHO cells. Glutaminolysis is the prevalent pathway through which CHO cells assimilate organic nitrogen for biomass synthesis while releasing ammonium as the main by-product. Particularly, the term ‘optimal amino acid metabolism’ refers to the ideal or best amino acid metabolism possible by a CHO cell.

The term ‘lipid metabolism’ as used herein refers to the synthesis and degradation of lipids in cells, involving the breakdown or storage of fats for energy and the synthesis of structural and functional lipids. Lipids are the major component of cellular membranes, act as secondary messengers in cell communication, involved in signalling, transport and secretion. Lipids are also an important source of energy through β-oxidation and the tricarboxylic acid (TCA) cycle. Lipid metabolism can have a significant impact on cell growth. For example, the process of triacylglycerol synthesis and degradation in CHO cells can greatly affect overall cellular metabolism and viability. Particularly, the term ‘optimal lipid metabolism’ refers to the ideal or best amino acid metabolism possible by a CHO cell.

Carbohydrate, amino acid and lipid metabolism can be determined by Metabolite Detection Assays, HPLC and bioprocess analyser. These methods are further disclosed at least in Coulet, M. et al., Cells (2022), 11, 1929; Fan Y, et al., Biotechnol Bioeng (2015) 112 (3): 521-535 and Ali A S, et al., Biotechnol J. (2018); 13 (10): e1700745.

The terms “methylation profile”, “methylation pattern”, “methylation state” or “methylation status,” are used herein to describe the state, situation or condition of methylation of a genomic sequence, and such terms refer to the characteristics of a DNA segment at a particular genomic locus in relation to methylation. Such characteristics include, but are not limited to, whether any of the cytosine (C) residues within this DNA sequence are methylated, location of methylated C residue(s), percentage of methylated C at any particular stretch of residues, and allelic differences in methylation due to, e.g., difference in the origin of the alleles.

The term “methylation status” refers to the status of a specific methylation site (i.e. methylated vs. non-methylated) which means a residue or methylation site is methylated or not methylated. Then, based on the methylation status of one or more methylation sites, a methylation profile may be determined. Accordingly, the term “methylation profile” or also “methylation pattern” refers to the relative or absolute concentration of methylated C residues or unmethylated C residues at any particular stretch of residues in the genomic material of a biological sample. For example, if cytosine (C) residue(s) not typically methylated within a DNA sequence are methylated, it may be referred to as “hypermethylated”; whereas if cytosine (C) residue(s) typically methylated within a DNA sequence are not methylated, it may be referred to as “hypomethylated”. Likewise, if the cytosine (C) residue(s) within a DNA sequence (e.g., the DNA from a sample nucleic acid from a test subject) are methylated as compared to another sequence from a different region or from a different individual (e.g., relative to normal nucleic acid or to the standard nucleic acid of the reference sequence), that sequence is considered hypermethylated compared to the other sequence. Alternatively, if the cytosine (C) residue(s) within a DNA sequence are not methylated as compared to another sequence from a different region or from a different individual, that sequence is considered hypomethylated compared to the other sequence. These sequences are said to be “differentially methylated”. Measurement of the levels of differential methylation may be done by a variety of ways known to those skilled in the art. One method is to measure the methylation level of individual interrogated CpG sites determined by the bisulfite sequencing method, as a non-limiting example.

The term “hypermethylation” refers to the average methylation state corresponding to an increased presence of 5-mCyt at one or a plurality of CpG dinucleotides within a DNA sequence of a test DNA sample, relative to the amount of 5-mCyt found at corresponding CpG dinucleotides within a normal control DNA sample.

The term “hypomethylation” refers to the average methylation state corresponding to a decreased presence of 5-mCyt at one or a plurality of CpG dinucleotides within a DNA sequence of a test DNA sample, relative to the amount of 5-mCyt found at corresponding CpG dinucleotides within a normal control DNA sample.

As used herein, a “methylated nucleotide” or a “methylated nucleotide base” refers to the presence of a methyl moiety on a nucleotide base, where the methyl moiety is usually not present in a recognized typical nucleotide base. For example, cytosine in its usual form does not contain a methyl moiety on its pyrimidine ring, but 5-methylcytosine contains a methyl moiety at position 5 of its pyrimidine ring. Therefore, cytosine in its usual form may not be considered a methylated nucleotide and 5-methylcytosine may be considered a methylated nucleotide. In another example, thymine may contain a methyl moiety at position 5 of its pyrimidine ring, however, for purposes herein, thymine may not be considered a methylated nucleotide when present in DNA. Typical nucleotide bases for DNA are thymine, adenine, cytosine and guanine. Typical bases for RNA are uracil, adenine, cytosine and guanine. Correspondingly a “methylation site” is the location in the target gene nucleic acid region where methylation has the possibility of occurring. For example, a location containing CpG is a methylation site wherein the cytosine may or may not be methylated. In particular, the term “methylated nucleotide” refers to nucleotides that carry a methyl group attached to a position of a nucleotide that is accessible for methylation. These methylated nucleotides are usually found in nature and to date, methylated cytosine that occurs mostly in the context of the dinucleotide CpG, but also in the context of CpNpG- and CpNpN-sequences may be considered the most common. In principle, other naturally occurring nucleotides may also be methylated but they will not be taken into consideration with regard to any aspect of the present invention.

“Reference methylation profiles” may be defined on the basis of multiple training samples using multivariate statistical methods, such as such as Principal Component analysis or Multi-Dimensional Scaling.

In particular, the reference methylation profile according to any aspect of the present invention is a compilation of more than one CpG site from at least one reference mammalian cell line that displays at least one phenotype of interest. In one example, the different CpG sites are collected from a single reference mammalian cell line that displays at least one phenotype of interest. In another example, the different CpG sites are collected from more than one cell line where each cell line displays at least one phenotype of interest. The reference methylation profile according to any aspect of the present invention may thus not be a naturally occurring methylation profile from a single mammalian cell line but an artificial profile obtained from combining relevant CpG sites from different reference mammalian cell lines, each with at least one phenotype of interest.

As used herein, a “CpG site” or “methylation site” is a nucleotide within a nucleic acid (DNA or RNA) that is susceptible to methylation either by natural occurring events in vivo or by an event instituted to chemically methylate the nucleotide in vitro. Some of these sites may be hypermethylated and some may be hypomethylated in a cell. In some cases a CpG site may not be considered fully hypermethylated or hypomethylated but a value may be given that is a measure of methylation of the CpG site. Accordingly, methylation may be quantified and may not always be an absolute case of hypermethylation or hypomethylation.

As used herein, a “methylated nucleic acid molecule” refers to a nucleic acid molecule that contains one or more nucleotides that is/are methylated.

A “CpG island” as used herein describes a segment of DNA sequence that comprises a functionally or structurally deviated CpG density. For example, Yamada et al. have described a set of standards for determining a CpG island: it must be at least 400 nucleotides in length, has a greater than 50% GC content, and an OCF/ECF ratio greater than 0.6 (Yamada et al., 2004, Genome Research, 14, 247-266). Others have defined a CpG island less stringently as a sequence at least 200 nucleotides in length, having a greater than 50% GC content, and an OCF/ECF ratio greater than 0.6 (Takai et al., 2002, Proc. Natl. Acad. Sci. USA, 99, 3740-3745).

In particular, when there is differential methylation detected in a test cell, that is to say that the cell displays absolute hypermethylation or hypomethylation or at least quantitative differential methylation at, at least one CpG site in comparison to the reference (i.e., from a CHO cell line with at least one phenotype of interest), then the test cell also comprises the phenotype of interest and may be capable of optimal heterologous protein production. More in particular, when the CpG site displays the same methylation status in the test cell in comparison to the corresponding CpG site in the reference cell or reference methylation profile, the test cell expresses the phenotype of interest and may be capable of optimal heterologous protein production. Overall, this platform gives us an opportunity to detect wide-spread DNA methylation status in CHO cells and correlate it with industrially relevant parameters which are crucial for the development of at least biological pharmaceutical products.

In particular, in the method according to any aspect of the present invention, in step (a) the methylation status of at least 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100 CpG sites are determined. A skilled person would be capable of determining the number of CpG sites that need to be used in step (a) according to any aspect of the present invention. Even more in particular, the methylation status of at least two CpG sites are determined in step (a) of the method according to any aspect of the present invention.

The term ‘epigenetic change’ as used herein refers to a chemical (e.g., methylation) change or protein (e.g., histones) change that takes place to a gene body or a promoter thereof. Through epigenetic changes, environmental factors like. diet, stress and prenatal nutrition can make an imprint on genes passed from one generation to the next.

As used herein, the term “significantly similar” refers to in particular in context with the comparison of methylation profiles (such as the comparison between test profiles (from test subject(s) and reference profiles) a similarity observed by statistical means (i.e. by using bioinformatics) and/or also by observation using the eye. A significant similarity is observed for example if a test profile overlaps with a reference profile that is defined by multiple training samples through multivariate statistical methods, such as Principal Component analysis or Multi-Dimensional Scaling. In particular, a test profile is significantly similar to the pre-determined reference profile if more than 50, 55, 60, 65, 70, 75, 80, 85, 90, 95% of the methylation pattern/profile overlaps with that of the reference profile. A similarity of a test profile to more than one, such as two, three or even all reference profile reduces the significance of the similarity.

As used herein, the term “genomic material” refers to nucleic acid molecules or fragments of the genome of the mammalian cells or cell lines. In particular, such nucleic acid molecules or fragments are DNA or RNA or hybrids thereof, and most preferably are molecules of the DNA genome of CHO cells or cell lines.

As used herein, the “DNA sample” refers to the DNA extracted from the cell according to any aspect of the present invention using known methods in the art.

As used herein, the term “pre-selected methylation sites” refers to methylation sites that were selected from genes or regions that showed the highest degree of methylation variation during the training of the method and fulfils certain quality criteria such as a minimum sequencing coverage of ≥5× were considered and for ≥5 qualified CpG sites. Additionally, genes that have an average methylation level <0.1 or an average methylation level >0.9 can be excluded due to their limited dynamic range. In particular, the pre-selected methylation sites is related to at least one phenotype of interest in the test cell line.

As used herein the term “cell culture medium” is used interchangeably with the term “cell medium” or fermentation broth, if the cell are cultured in a fermenter or bioreactor. In particular, the cell medium refers to a medium to culture mammalian cells comprising a minimum of essential nutrients and components such as vitamins, trace elements, salts, bulk salts, amino acids, lipids, carbohydrates in a particularly buffered medium (for example with a pH about 7.0, particularly with a pH=7.3-6.6, more particularly with a pH of 7.0). The cell culture medium may be a basal cell culture medium or a basal cell culture medium to which additives may be added. Any ingredient and/or additive of the culture medium may be considered a “component” of the cell medium as the array according to any aspect of the present invention is developed from experimental based functional CpG sites.

The term “basal medium” or “basal cell culture medium” as used herein is a cell medium to culture mammalian cells and where the medium is used to culture the cells from the start of a cell culture run and is not used as an additive to another medium, although various (test) components may be added to the medium. The basal medium serves as the base to which optionally further additives or feed medium may be added during cultivation, i.e., a cell culture run. The basal cell culture medium is provided from the beginning of a cell cultivation process. In general, the basal cell culture medium provides nutrients such as carbon sources, amino acids, vitamins, bulk salts (e.g. sodium chloride or potassium chloride), various trace elements (e.g. manganese sulfate), pH buffer, lipids and glucose. Major bulk salts are usually provided only in the basal medium and must not exceed a final osmolarity in the cell culture of about 280-350 mOsm/kg, so that the cell culture is able to grow and proliferate at a reasonable osmotic stress.

The term “feed” or “feed medium” as used herein relates to a concentrate of nutrients/a concentrated nutrient composition used as a feed in a culture of mammalian cells. It is provided as a “concentrated feed medium” to avoid dilution of the cell culture. A feed medium typically has higher concentrations of most, but not all, components of the basal cell culture medium. Generally, the feed medium substitutes nutrients that are consumed during cell culture, such as amino acids and carbohydrates, while salts and buffers are of less importance and are commonly provided with the basal medium. The feed medium is typically added to the (basal) cell culture medium/fermentation broth in fed-batch mode. However, the feed may be added in different modes like continuous or bolus addition or via perfusion related techniques (chemostat or hybrid-perfused system). Each of the ingredients, specific concentration of each of the ingredients of the feed or feed medium may fall within the definition of “test component” as used herein. The feeding rate is to be understood as an average feeding rate over the feeding period. Particularly, the feed medium is added daily, but may also be added more frequently, such as twice daily or less frequently, such as every second day. The addition of nutrients is commonly performed during cultivation (i.e., after day 0). In contrast to the basal medium, the feed consists of a highly concentrated nutrient solution (e.g. >6×) that provides all the components similar to the basal medium except for ‘high-osmolarity-active compounds’ such as major bulk salts (e.g., NaCl, KCl, NaHC0₃, MgS0₄, Ca(N0₃)₂).

The cell culture medium, both basal medium and/or feed medium may be serum-free, chemically defined or chemically defined and protein-free. A “serum-free medium” as used herein refers to a cell culture medium for in vitro cell culture, which does not contain serum from animal origin. This is preferred as serum may contain contaminants from said animal, such as viruses, and because serum is ill-defined and varies from batch to batch. The basal medium and the feed medium according to any aspect of the present invention may be serum-free.

A “chemically defined medium” as used herein refers to a cell culture medium suitable for in vitro cell culture, in which all components are known. More specifically it does not comprise any supplements such as animal serum or plant, yeast or animal hydrolysates. It may comprise hydrolysates only if all components have been analysed and the exact composition thereof is known and can be reproducibly prepared. The basal medium and the feed medium according to the invention are preferably chemically defined.

The term “commercially available media/media systems” as used herein refers to commercially available cell culture media with completely known composition. These media serve as references for the media of the present invention due to the requirement for exact nutrient composition. Commercially available media are, e.g., DMEM:F12 (1:1), DMEM, HamsF12, and RPMI. The feed medium of the commercial media used herein were prepared as a 12-fold concentrate of the basal medium without bulk salts. The term “commercially available media systems” relate to a system comprising of a commercially available basal cell culture medium, such as DMEM:F12 (1:1), DMEM, HamsF12, and RPMI and a feed medium, which is the respective concentrated basal medium (e.g., 12-fold concentrated) without or with reduced bulk salts.

The term “cell medium” according to any aspect of the present invention, may refer to any one of the above cell media for mammalian cell culture. A test component may then be added to the cell medium and the effect of the component on the mammalian cell determined using the method according to any aspect of the present invention. In particular, the component added to the cell medium may be selected from the group consisting of amino acids, small peptides, buffering agents, a carbon-based energy source, such as carbohydrates (e.g. glucose, mannose, etc.), inorganic salts or ions, serum (or its essential components, including growth factors, hormones, lipids, proteins, and trace elements), vitamins and minerals.

The term “amino acid” as used herein refers to the twenty natural amino acids that are encoded by the universal genetic code, typically the L-form (i.e., L-alanine, L-arginine, L-asparagine, L-aspartic acid, L-cysteine, L-glutamic acid, L-glutamine, L-glycine, L-histidine, L-isoleucine, L-leucine, L-lysine, L-methionine, L-phenylalanine, L-proline, L-serine, L-threonine, L-tryptophan, L-tyrosine and L-valine). The amino acids (e.g., glutamine and/or tyrosine) may be provided as dipeptides with increased stability and/or solubility, preferably containing an L-alanine (L-ala-x) or L-glycine extension (L-gly-x), such as glycyl-glutamine and alanyl-glutamine. Further, cysteine may also be provided as L-cystine. The term “amino acids” as used herein encompasses all different salts thereof, such as L-arginine monohydrochloride, L-asparagine monohydrate, L-cysteine hydrochloride monohydrate, L-cystine dihydrochloride, L-histidine monohydrochloride dihydrate, L-lysine monohydrochloride and hydroxyl L-proline, L-tyrosine disodium dehydrate.

Suitable buffering agents include, but are not limiting to Hepes, phosphate buffers (e.g., potassium phosphate monobasic and potassium phosphate dibasic and/or sodium phosphate dibase anhydrate and sodium phosphate monobase), phenol red, sodium bicarbonate and/or sodium hydrogen carbonate.

The term “cell cultivation” or “cell culture” includes cell cultivation and fermentation processes in all scales (e.g. from micro titre plates to large-scale industrial bioreactors, i.e. from sub mL-scale to >10.000 L scale), in all different process modes (e.g. batch, fed-batch, perfusion, continuous cultivation), in all process control modes (e.g. non-controlled, fully automated and controlled systems with control of e.g. pH, temperature, oxygen content), in all kind of fermentation systems (e.g. single-use systems, stainless steel systems, glass ware systems). In a preferred embodiment of the present invention the cell culture is a mammalian cell culture and is a batch or a fed-batch culture.

The term “fed-batch” as used herein relates to a cell culture in which the cells are fed continuously or periodically with a feed medium containing nutrients. The feeding may start shortly after starting the cell culture on day 0 or more typically one, two or three days after starting the culture. Feeding may follow a pre-set schedule, such as every day, every two days, every three days etc. Alternatively, the culture may be monitored for cell growth, nutrients or toxic by-products and feeding may be adjusted accordingly. Common monitoring methods for animal cell culture are described in the experimental part below. In general, the following parameters are often determined on a daily basis and cover the viable cell concentration, product concentration and several metabolites such as glucose or lactic acid (an acidic waste metabolite that reduces the pH and is derived from cellular glucose conversion), pH, osmolarity (a measure for salt content) and ammonium (growth inhibitor that negatively affects the growth rate and reduces viable biomass). Compared to batch cultures (cultures without feeding), higher product titres can be achieved in the fed-batch mode. Typically, a fed-batch culture is stopped at some point and the cells and/or the protein of interest in the medium are harvested and optionally purified.

The term “test” used in conjunction with the term cell herein refers to an entity that is subjected to the method according to any aspect of the present invention and is the basis for an analysis application of the present invention. A “test cell” or a “test profile” is therefore a cell being tested according to the invention or a profile being obtained or generated in this context. Conversely, the term “reference” shall denote, mostly predetermined, entities which are used for a comparison with the test entity. For example, the term ‘reference cell refers to a cell used for comparison or as a control in reference to the ‘test cell. Similarly, the term ‘sample’ and/or ‘test cell DNA sample’ used in accordance with any aspect of the present invention refers to an entity that may be subject to the method according to any aspect of the present invention. In particular, a sample may be any DNA sample obtained from a test cell that may be subject to the method according to any aspect of the present invention to determine the effect of a selected component of the cell on the phenotype of interest of the cell by first determining the DNA methylation profile and then comparing this test methylation profile with a control (reference methylation profiles from control cells showing or not showing a phenotype of interest).

As used herein, the term “comprising” is to be construed as encompassing both “including” and “consisting of”, both meanings being specifically intended, and hence individually disclosed aspects of the present invention. Where used herein, “and/or” is to be taken as specific disclosure of each of the two specified features or components with or without the other. For example, “A and/or B” is to be taken as specific disclosure of each of (i) A, (ii) B and (iii) A and B, just as if each is set out individually herein. In the context of the present invention, the terms “about” and “approximately” denote an interval of accuracy that the person skilled in the art will understand to still ensure the technical effect of the feature in question. The term typically indicates deviation from the indicated numerical value by ±20%, ±15%, ±10%, and for example ±5%. As will be appreciated by the person of ordinary skill, the specific deviation for a numerical value for a given technical effect will depend on the nature of the technical effect. For example, a natural or biological technical effect may generally have a larger such deviation than one for a man-made or engineering technical effect. Where an indefinite or definite article is used when referring to a singular noun, e.g. “a”, “an” or “the”, this includes a plural of that noun unless something else is specifically stated.

The term ‘performance’ as used herein refers to the protein production ability of the cell (i.e. phenotypic homogeneity and protein productivity, and protein quality). The term ‘general stability’ of the cell refers to the status of the cell's survivability, viability, vitality, cell exhaustion and the like.

The terms “vitality” and “viability” are used interchangeably and refers to the % viable cells in a cell culture as determined by methods known in the art, e.g., trypan blue exclusion with a Cedex device based on an automated-microscopic cell count (Innovatis AG, Bielefeld). However, there exist of number of other methods for the determination of the viability such as fluorometric (such as based on propidium iodide), calorimetric or enzymatic methods that are used to reflect the energy metabolism of a living cell e.g. methods that use LDH lactate-dehydrogenase or certain tetrazolium salts such as alamar blue, MTT (3-(4,5-dimethylthiazol-2-yl-2,5-diphenyltetrazolium bromide) or TTC (tetrazolium chloride).

A “mammalian cell” as used herein refers to is a cell from any member of the order Mammalia which includes a cell from a mouse, a rat, a monkey, a guinea pig, a dog, a mini-pig, a human being, a cow, a sheep, a pig, a goat, a horse, a donkey, a mule, a hamster, a cat, a dolphin, an elephant or the like. The mammalian cell may also include an established cell line or immortalized cell line. In particular, the immortalised cell line may be capable of protein, specifically therapeutic protein production. More in particular, the immortalized cell line may be a therapeutic immortalised cell line. For example, the mammalian cell according to any aspect of the present invention may be a CHO cell line which refers to immortal Chinese Hamster Ovary cell line (CHO) derived from Cricetulus griseus. In particular, the CHO cell line may be selected from the group consisting of CHO-K1 (ATCC), CHO-DG44 (Thermo Fisher Scientific), CHO-DXB11 (ATCC), ExpiCHO-S™ cells (Thermo Fisher Scientific), FreeStyle™ CHO-S™ cells (Thermo Fisher Scientific), CHO 1-15 [subscript 500] (ATCC), Agarabi CHO (ATCC), and a CHOK1SV cell including all variants (e.g. POTELLIGENT®, Lonza, Slough, UK), a CHOK1SV GS-KO (glutamine synthetase knockout) cell including all variants (e.g., XCEED™ Lonza, Slough, UK). The mammalian cell may be from Baby Hamster Kidney fibroblasts (BHK (ATCC CCL-10), or Vero cell (ATCC CCL-81). Exemplary human cells include human embryonic kidney (HEK) cells, such as HEK293 (ATCC CRL-1573), HEK 293T (ATCC CRL-3216), a HeLa cell (ATCC CCL-2), a NS0 cell (ECACC 85110503), or a Sp2/0 cell (ATCC CRL-1581). The mammalian cells according to any aspect of the present invention may include mammalian cell cultures which can be either adherent cultures or suspension cultures.

The method according to any aspect of the present of the present invention is a DNA-based array, particularly a DNA-methylation based array. Arrays allow for a high-throughput and robust method to determine semi-quantitative/quantitative DNA-methylation information through a small sample of extracted DNA of interest. These custom designed arrays may use Illumina iScan and Infinium platform technology or an equivalent thereof, which allows on each chip for example 100,000 different bead types that covalently bind DNA-methylation probes. Each probe represents one CpG Methylation site at the end of the probe sequence. DNA samples undergo bisulfite conversion, amplification, fragmentation, precipitation and resuspension steps before hybridization on an array chip. Once on the chip the DNA hybridizes to the beads for each CpG site so that methylation changes at each site can be detected specifically through single nucleotide extension. This is especially advantageous as the array-based method is simple and the results of the array are accurate and reproducible. Compared to other methods in the art where WGS/WGBS is conducted to identify differential methylation, the customized DNA methylation-based array according to any aspect of the present invention may be used to assess DNA methylation making the method according to any aspect of the present invention more efficient and accurate compared to those known in the art. In particular, the DNA methylation-based array according to any aspect of the present invention is based on the deduction of methylation values from multiple CpG sites across the CHO cell genome (i.e. Differentially Methylated Regions, Dynamic regions, Variably methylated regions) and regulatory regions in the CHO cell genome.

Further, compared to traditional sequencing which can take weeks to generate data, the array technology has a much shorter turn-around time. The volume and complexity of data generated is lesser compared to sequencing making it computationally less intensive. This allows for quicker computation to achieve interpretable results from experimental groups. Overall microarray technology is roughly 10× faster and 10× cheaper than traditional sequencing while still quantifiable for the methylation level at specific CpG sites.

The term “array” as used herein refers to an intentionally created collection of probe molecules which can be prepared either synthetically or biosynthetically. The probe molecules in the array can be identical or different from each other. The array can assume a variety of formats, for example, libraries of soluble molecules; libraries of compounds tethered to resin beads, silica chips, or other solid supports.

In particular, an array provides a convenient platform for simultaneous analysis of large numbers of CpG sites, for example, at least 2, 3, 4, 5, 6, 7, 8, 9, 10, 50, 100, 500, 1000, 5000, 10,000, 100,000 or more sites or loci. In particular, the array comprises a plurality of different probe molecules that can be attached to a substrate or otherwise spatially distinguished in an array. Examples of arrays that may be used according to any aspect of the present invention include slide arrays, silicon wafer arrays, liquid arrays, bead-based arrays and the like. In one example, array technology used according to any aspect of the present invention combines a miniaturized array platform, a high level of assay multiplexing, and scalable automation for sample handling and data processing.

In particular, the array according to any aspect of the present invention may be an array of arrays, also referred to as a composite array, having a plurality of individual arrays that is configured to allow processing of multiple samples simultaneously. Examples of composite arrays and the technology behind them are disclosed at least in U.S. Pat. No. 6,429,027 and US 2002/0102578. A substrate of a composite array may include a plurality of individual array locations, each having a plurality of probes, and each physically separated from other assay locations on the same substrate such that a fluid contacting one array location is prevented from contacting another array location. Each array location can have a plurality of different probe molecules that are directly attached to the substrate or that are attached to the substrate via rigid particles in wells (also referred to herein as beads in wells).

In one example, an array substrate can be a fibre optical bundle or array of bundles as described in U.S. Pat. Nos. 6,023,540, 6,200,737 and/or 6,327,410. An optical fibre bundle or array of bundles can have probes attached directly to the fibres or via beads. A skilled person would be able to easily determine which substrate will be most suitable for the array according to any aspect of the present invention. WO2004110246 further discloses other substrates and methods of attaching beads to the substrates that may be used in the array according to any aspect of the present invention.

In one example, a surface of the substrate may have physical alterations to enable the attachment of probes or produce array locations. For example, the surface of a substrate can be modified to contain chemically modified sites that are useful for attaching, either-covalently or non-covalently, probe molecules or particles having attached probe molecules. Probes may be attached using any of a variety of methods known in the art including, an ink-jet printing method, a spotting technique, a photolithographic synthesis method, or printing method utilizing a mask. WO2004110246 discloses these techniques in more detail.

In one example, the array according to any aspect of the present invention may be a bead-based array, where the beads are associated with a solid support such as those commercially available from Illumina, Inc. (San Diego, Calif.). An array of beads useful according to any aspect of the present invention can also be in a fluid format such as a fluid stream of a flow cytometer or similar device. Commercially available fluid formats for distinguishing beads include, for example, those used in XMAP™ technologies from Luminex or MPSS™ methods from Lynx Therapeutics.

The term “solid support”, “support”, and “substrate” as used herein are used interchangeably and refer to a material or group of materials having a rigid or semi-rigid surface or surfaces. In many examples, at least one surface of the solid support will be substantially flat, although in some examples it may be desirable to physically separate synthesis regions for different compounds with, for example, wells, raised regions, pins, etched trenches, or the like.

The array or microarray according to any aspect of the present invention may be a very high-density array, for example, those having from about 10,000,000 probes/cm² to about 2,000,000,000 probes/cm² or from about 100,000,000 probes/cm² to about 1,000,000,000 probes/cm². High density arrays are especially useful according to any aspect of the present invention for including the multitude of CpG sites on the array.

The array according to any aspect of the present invention may be used to analyse or evaluate such pluralities of loci simultaneously or sequentially as desired. In one example, a plurality of different probe molecules can be attached to a substrate or otherwise spatially distinguished in an array. Each probe is typically specific for a particular locus and can be used to distinguish methylation state of the locus.

The term “probe molecules” or ‘probes’ as used interchangeably herein refers to a surface-immobilized molecule that can be recognized by a particular target. Probes used in the array can be specific for the methylated allele of a CpG site, the non-methylated allele of the CpG site or both or for the methylated allele of a non-CpG site, the non-methylated allele of the non-CpG site or both.

The term “target” as used herein refers to a molecule that has an affinity for a given probe molecule. Targets may be naturally occurring or man-made molecules. Also, they can be employed in their unaltered state or as aggregates. Targets may be attached, covalently or noncovalently, to a binding member, either directly or via a specific binding substance. Examples of targets which can be employed according to any aspect of the present invention are methylated and non-methylated CpG sites. Targets are sometimes referred to in the art as anti-probes. As the term targets is used herein, no difference in meaning is intended.

The term “complementary” as used herein refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Perfectly complementary refers to 100% complementarity over the length of a sequence. For example, a 25-base probe is perfectly complementary to a target when all 25 bases of the probe are complementary to a contiguous 25 base sequence of the target with no mismatches between the probe and the target over the length of the probe.

The method according to any aspect of the present invention comprises a further step of:
(c) comparing the test methylation profile obtained from (a) with
   (i) at least one first reference methylation profile obtained from a first mammalian reference cell line that displays at least one phenotype of interest; and/or
   (ii) at least one second reference methylation profile obtained from a second mammalian reference cell line that does not display the phenotype of interest; and
wherein the reference cell lines are not in contact with the test component; and
wherein a significant similarity in the test methylation profile of (a) compared to the first or second reference methylation profile, is indicative of the test cell having the phenotype of interest or not having the phenotype of interest respectively; and
wherein a difference in the test methylation profile of (a) compared to the first or second reference methylation profile, is indicative of the test cell not having the phenotype of interest or having the phenotype of interest.

In particular, the first reference methylation profile is a compilation of more than one CpG site from at least one reference cell line that displays at least one phenotype of interest; and the second reference methylation profile is a compilation of more than one CpG site from at least one reference cell line that does not display at least one phenotype of interest.

The reference methylation profiles, particularly the first and second reference methylation profiles, are “pre-determined reference profiles” used to refer to a typical or standard methylation profile of the genomic material of a mammalian reference cell line that displays at least one phenotype of interest. In one example, the pre-determined reference profile may be used in the context of a control cell, where the control cell has exhibited good protein production (i.e. the control cell is capable of high quantitative and qualitative protein production). In particular, the term “pre-determined reference profile” herein may be used in the context of a control cell, where the control animal has good protein production and/or general stability wherein the control cell has optimal carbohydrate metabolism, optimal amino acid metabolism, optimal lipid metabolism, optimal cell survivability and combinations thereof compared to baseline values of a cell of the same species as the control cell.

As used herein, the term “baseline” relative to phenotype of interest refers to various aspects of a cell when the cell is cultured in a cell medium without one or more optional supplements. That is to say, the phenotype of interest when the cell is cultured in a basal medium. A panel of pre-determined reference profiles for control cells may also include profiles from different samples that exhibit different phenotypes of interests or combinations thereof. Each of these samples may have its own unique pre-determined methylation reference profile that also forms a part of the panel of pre-determined reference profiles.

According to a further aspect of the present invention, there is provided a DNA array-based method of assessing the effect of at least one test component of cell media on the production of at least one biosimilar from an immortalised test cell line,

wherein the biosimilar is significantly similar relative to an innovator protein produced by an immortalized reference cell line which is the same cell line as the test cell line, the method comprising the steps of:
(a) determining a first test methylation profile from DNA obtained from the immortalized test cell line that is cultured in the cell media comprising the test component, and
(b) determining a second methylation profile from DNA obtained from the immortalized test cell line that is cultured in the test component absent cell media;
(c) comparing the test methylation profile obtained from (a) and (b) with a reference methylation profile obtained from a immortalized reference cell line;
   wherein a significant similarity between the test methylation profile of (a) and the reference methylation profile and a difference between the test methylation profile of (b) and the reference methylation profile is indicative of the two cell lines producing biosimilars and the test component having a positive effect on the production of biosimilars from immortalised cell lines.

The term ‘biosimilar’ as used herein refers to recombinant proteins produced by genetically modified mammalian cells which are highly similar to the original biotherapeutic reference product and share quality, safety and efficacy with the reference product. In particular, the product produced is phenotypically/epigenetically similar to the reference product. The term ‘biosimilar’ is more clearly explained at least in A. Ishii-Watabe, et al., (2019) Drug Metab. Pharmacokinet. 34 (1): 64-70 and Wolff-Holz, E., et al., (2019) BioDrugs 33, 621-634.

Information on DNA methylation patterns for cell lines could result in a clearer specification profile for product release in mammalian cells and could serve as a “copyright” protection from biosimilar developers, and could develop as potential “gold standard”, for the regulatory process required for biosimilar development.

The term “innovator protein” used herein refers to the wild-type protein, the protein that is found in nature.

According to yet a further aspect of the present invention, there is provided a DNA array-based method of assessing the effect of at least one test component of cell media on the production of at least one bioidentical from an immortalised test cell line, wherein the bioidentical is significantly similar relative to an innovator protein produced by an immortalized reference cell line which is the same cell line as the test cell line, the method comprising the steps of:
(a) determining a first test methylation profile from DNA obtained from the immortalized test cell line that is cultured in the cell media comprising the test component, and
(b) determining a second methylation profile from DNA obtained from the immortalized test cell line that is cultured in the test component absent cell media;
(c) comparing the test methylation profile obtained from (a) and (b) with a reference methylation profile obtained from a immortalized reference cell line;
   wherein a significant similarity between the test methylation profile of (a) and the reference methylation profile and a difference between the test methylation profile of (b) and the reference methylation profile is indicative of the two cell lines producing bioidenticals and the test component having a positive effect on the production of bioidenticals from immortalized cell lines.

As used herein, the term ‘bioidentical’ refers to recombinant proteins produced by genetically modified mammalian cells that have the same molecular structure as the original biotherapeutic reference product. The term ‘bioidentical’ is more clearly explained at least in Stanczyk F Z, et al., Climacteric. 2021; 24:38-45.

Mammalian cells, particularly CHO cells, that are able to produce biosimilar or bioidentical proteins have a significantly similar or identical CpG methylation profile respectively to a reference profile from a mammalian cell of the same type as the test mammalian cell, particularly a parental clone that is capable of producing proteins most similar to the wildtype protein, particularly therapeutic protein. In another example, mammalian cell that produce biosimilar or bioidentical proteins have a significantly similar or identical methylation profile of a selected region (e.g. but not restricted to low methylated regions (LMR)/partially methylated domains (PMD)/differentially methylated regions (DMR)/differentially methylated points (DMP) to a reference profile from a mammalian cell, particularly a parental clone that is capable of producing proteins most similar to the wildtype protein, particularly therapeutic protein. In another example, the mammalian cell that produce biosimilar or bioidentical proteins have a significantly higher CpG Methylation distribution (e.g., beta value distribution) compared to other mammalian cells. In yet another example, a mammalian cell that produce biosimilar or bioidentical proteins has no or the least amount of partial methylation at each site compared to other cells. In particular, the heterologous protein is a monoclonal antibody and/or therapeutic protein.

Low Methylated Region (LMR) is a region of the genome wherein less than 60% of CpGs in that region are methylated. More in particular, less than 50%, 40%, 30%, 20% or 10% of the CpGs in the LMRs are methylated. Any method known in the art may be used to identify or detect LMRs in the genomic DNA. Well known methods include using programmes such as MethylSeekR. In particular, LMRs in the genomic DNA have at least three consecutive CpGs and have no single nucleotide polymorphisms (SNPs) in any of the CpG positions. Even more in particular, LMRs in the genomic DNA are identified based on the method disclosed at least in Burger, L., (2013) Nucleic Acids Research, 41 (16): e155 and/or Stadler, M., (2011) Nature 480, 490-495. LMRs are known to have an average methylation ranging from 10% to 50%; are regions of low CG density which do not overlap with CpG islands; tend to be enriched for H3K4me1, DHSs, and p300/CBP; and/or are primarily located distal to promoters in intergenic or intronic regions. In particular, LMRs:
have an average methylation ranging from 10% to 50%,
are regions of low CG density;
are enriched for Histone H3 monomethylated at lysine 4 (H3K4me1), DNase I hypersensitive sites (DHSs) and transcriptional coactivators CREB binding protein (CPB) and p300;
are primarily located distal to promoters in intergenic or intronic regions; and/or
have no single nucleotide polymorphisms (SNPs) in any of the CpG positions.

Low-methylated regions (LMRs) represent a key feature of the dynamic methylome. LMRs are local reductions in the DNA methylation landscape and represent CpG-poor distal regulatory regions that often reflect the binding of transcription factors and other DNA-binding proteins. LMRs were originally described in the mouse (Stadler et al. (2011) Nature: 480, 490-95). Evolutionary conservation of LMRs beyond mammals has remained unexplored.

Differentially methylated regions (DMRs) are genomic regions with different methylation statuses among multiple biological samples like tissues, cells, individuals, etc. These are genomic regions that differ between phenotypes. The statistical power is likely to be greater when adjacent DMPs are considered together as a whole [Gu H et al (2010) Nat Methods 2010; 7:133-6]. The lengths of the DMRs may range between a few hundred to a few thousand bases [Rakyan et al (2011) Nat Rev Genet 12:529-41, 2011, Bock C (2012) Nat Rev Genet 2012; 13:705-19].

DMRs may occur throughout the genome but have been identified particularly around the promoter regions of genes, within the body of genes, and at intergenic regulatory regions. There are two types of regions, predefined or user defined. Regions with special biological meaning, such as CpG islands, CpG shores, UTRs and so on, are predefined. Many traditional statistical testings, including t-test and Wilcoxon rank sum test, can be performed at a region level. For user-defined regions, criteria such as a fixed region length, fixed numbers of significant and adjacent CpG sites, significant and smoothed estimated effect sizes, etc.

Partially methylated domains (PMDs) are extended regions in the genome exhibiting a reduced average DNA methylation level. They cover gene-poor and transcriptionally inactive regions and tend to be heterochromatic.

Differentially methylated Positions (DMP) are CpG sites with different DNA methylation status across different biological samples and regarded as possible functional regions involved in gene transcriptional regulation.

According to a further aspect of the present invention, there is provided a use of a DNA-methylation based array for determining the effect of at least one test component of cell media on producing mammalian cell lines displaying at least one phenotype of interest.

According to yet a further aspect of the present invention, there is provided a DNA methylation-based array for determining the effect of at least one test component of cell media on producing mammalian cell lines displaying at least one phenotype of interest.

According to another aspect of the present invention, there is provided a method for developing a DNA array-based test system for determining if a test component of cell media can produce a test mammalian cell line that is capable of optimal heterologous protein production, the method comprising the steps of:
(a) determining a first test methylation status of one or more pre-selected methylation sites from the genomic material obtained from the test CHO cell line cultured in cell media comprising the test component;
(b) determining a second test methylation status of one or more pre-selected methylation sites from the genomic material obtained from the test CHO cell line cultured in cell media absent of the test component;
(c) selecting from the pre-selected methylation sites a reference panel of methylation sites which is characterized by a specific and distinct differential methylation profile for each phenotypic parameter or phenotype of interest;
(d) obtaining a test system by assigning a reference methylation profile for each of the phenotypic parameter or phenotypes of interest; and
   wherein a comparison of a test methylation profile obtained from (a) and (b) with the reference methylation profiles obtained in (c) allows for confirming if the test mammalian cell line is capable of optimal heterologous protein production and if the test component has a positive, negative or no effect on the mammalian cell lines capability of optimal heterologous protein production.

### EXAMPLES

The foregoing describes preferred embodiments, which, as will be understood by those skilled in the art, may be subject to variations or modifications in design, construction or operation without departing from the scope of the claims. These variations, for instance, are intended to be covered by the scope of the claims.

### Example 1

### Oxidative Stress in CHO Cell Culture

### Wet-Lab Methodology

For this experiment, a transgenic CHO cell line, Agarabi CHO (ATCC® CRL-3440™), was grown in CD FortiCHO medium supplemented with 8 mM L-glutamine at 37° C., 8% CO2, at a shaking speed of 130 RPM. Batch culture of 6 flasks was maintained for 7 days where 3 flasks represent technical replicates for the control set and 3 flasks represent technical replicates for the treatment set. The flasks were seeded with 3E5 viable cells/mL on day 0 and to induce oxidative stress, hydrogen peroxide was added every 48 hrs to the treatment set with a final concentration of 120 μM. Cell count, cell viability, and heterologous protein production were measured every 2 days and cell pellets were collected for both control and treatment set on day 7. Induction of oxidative stress in CHO cells by treatment with hydrogen peroxide resulted in reduced growth rate and cell viability compared to control set and thus there was a slight increase in heterologous protein productivity for treatment set.

Genomic DNA was purified from the collected cell pellets using DNeasy Blood & Tissue Kit (Qiagen) and was quantified using PicroGreen or NanoDrop™ 2000. The genomic DNA (500 ng) from the control and treatment set were used to prepare libraries for Whole Genome Bisulfite Sequencing (WGBS). The sequencing of the libraries was performed by a third party on a NovaSeq platform which generated 125 GB of data per sample.

### Computational Methodology

Raw sequencing data were conducted quality control (fastqc) 1, sequencing adaptors trimming (TrimGalore) 2, and alignment with Bismark3. CMV promoter combined with CHOK1-GS (Cricetulus griseus) genome was used as a reference genome.

Bismark was also used for removing duplicated reads and extracting methylation counts from alignment output. SNPs were filtered out, and only counts with a minimum coverage of 10× were used for the downstream analysis, which resulted in 3711013 CpG sites for hydrogen peroxide treatment samples.

Since regulated methylation targets are most commonly clustered into short regions, DMRfinder4 was used to perform a modified single-linkage clustering of methylation sites. With a maximum distance between CpG sites of 100 bp, 1728014 genomic regions were found for hydrogen peroxide treatment samples.

### Differential Methylation Analysis

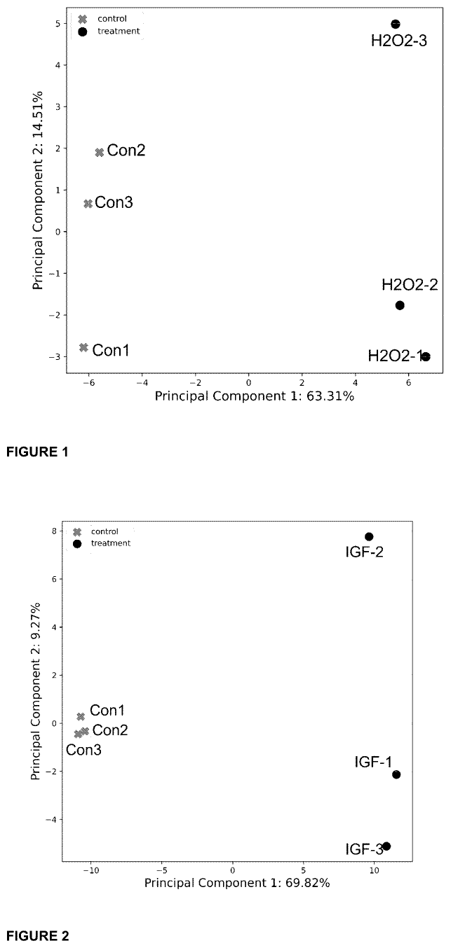

Differential methylation analysis was performed using MethylKit5 between the control and treatment groups. Logistic regression was used to determine the differential methylation across all regions, and the sliding linear model (SLIM) 6 method to do FDR correction. Regions with FDR corrected p-value <0.05 and methylation change greater than 25% between groups were determined as differentially methylated regions (DMRs), which were 122 for hydrogen peroxide treatment samples, shown in Table 1. Principal Component Analysis (PCA) is a dimensionality reduction technique that emphasizes variation in a dataset. PCA analysis for DMRs is shown in FIG. 1.

Preliminary results show DMRs play roles in epigenetic changes of oxidative stress, which can be potentially used as markers for future research.

TABLE 1
List of differentially methylated regions (DMRs) identified in hydrogen peroxide treatment samples

| chr | start | end |
|---|---|---|
| scaffold_11 | 28709706 | 28710166 |
| scaffold_37 | 2633223 | 2633699 |
| scaffold_3 | 51648552 | 51648916 |
| scaffold_5 | 69834725 | 69835199 |
| scaffold_8 | 5552117 | 5552561 |
| scaffold_26 | 21297189 | 21297609 |
| scaffold_3 | 89611520 | 89611822 |
| scaffold_22 | 26009928 | 26010211 |
| scaffold_5 | 71575709 | 71576056 |
| scaffold_1 | 39368692 | 39369030 |
| scaffold_5 | 18517703 | 18518011 |
| scaffold_29 | 14434642 | 14434837 |
| scaffold_30 | 17539176 | 17539422 |
| scaffold_35 | 1506683 | 1507183 |
| scaffold_2 | 33171492 | 33171654 |
| scaffold_29 | 21214834 | 21215287 |
| scaffold_31 | 7044527 | 7044866 |
| scaffold_4 | 36086102 | 36086595 |
| scaffold_67 | 1370390 | 1370856 |
| scaffold_35 | 11121893 | 11122057 |
| scaffold_38 | 12852538 | 12852906 |
| scaffold_1 | 39315830 | 39316127 |
| scaffold_0 | 34089755 | 34090116 |
| scaffold_6 | 72951899 | 72952171 |
| scaffold_92 | 551040 | 551276 |
| scaffold_12 | 35346308 | 35346549 |
| scaffold_7 | 66793870 | 66793964 |
| scaffold_8 | 5881102 | 5881479 |
| scaffold_5 | 27353205 | 27353427 |
| scaffold_6 | 38750584 | 38750737 |
| scaffold_19 | 29152850 | 29152892 |
| scaffold_10 | 44249680 | 44249959 |
| scaffold_31 | 19208325 | 19208599 |
| scaffold_0 | 89742694 | 89743065 |
| scaffold_22 | 10120022 | 10120299 |
| scaffold_5 | 75352416 | 75352711 |
| scaffold_17 | 22318231 | 22318310 |
| scaffold_2 | 64964306 | 64964784 |
| scaffold_27 | 2573631 | 2573958 |
| scaffold_0 | 126101885 | 126101986 |
| scaffold_27 | 7559618 | 7559924 |
| scaffold_0 | 141397264 | 141397482 |
| scaffold_15 | 11750842 | 11751221 |
| scaffold_18 | 18899911 | 18900021 |
| scaffold_31 | 11436108 | 11436414 |
| scaffold_22 | 11319316 | 11319522 |
| scaffold_10 | 7547985 | 7548213 |
| scaffold_29 | 19957756 | 19958066 |
| scaffold_13 | 1557582 | 1557813 |
| scaffold_31 | 216291 | 216630 |
| scaffold_18 | 1845104 | 1845449 |
| scaffold_6 | 47899988 | 47900353 |
| scaffold_2 | 3574161 | 3574486 |
| scaffold_22 | 19696673 | 19696913 |
| scaffold_2 | 27271033 | 27271300 |
| scaffold_48 | 589774 | 590045 |
| scaffold_3 | 54596703 | 54597029 |
| scaffold_0 | 119459381 | 119459708 |
| scaffold_22 | 557058 | 557163 |
| scaffold_17 | 20092773 | 20093109 |
| scaffold_0 | 175572250 | 175572386 |
| scaffold_27 | 4544681 | 4544868 |
| scaffold_3 | 35596025 | 35596105 |
| scaffold_38 | 7029674 | 7029838 |
| scaffold_16 | 6569320 | 6569338 |
| scaffold_31 | 13557315 | 13557751 |
| scaffold_22 | 27111725 | 27111786 |
| scaffold_24 | 31162074 | 31162369 |
| scaffold_2 | 23082378 | 23082645 |
| scaffold_0 | 172352541 | 172352799 |
| scaffold_3 | 117557801 | 117558116 |
| scaffold_1 | 53974390 | 53974471 |
| scaffold_31 | 17314786 | 17315000 |
| scaffold_31 | 15964682 | 15964838 |
| scaffold_3 | 25998505 | 25998572 |
| scaffold_35 | 13279321 | 13279421 |
| scaffold_9 | 14408479 | 14408926 |
| scaffold_9 | 22223538 | 22223924 |
| scaffold_2 | 15803480 | 15803773 |
| scaffold_22 | 31474980 | 31475143 |
| scaffold_100 | 220811 | 221203 |
| scaffold_0 | 28634272 | 28634445 |
| scaffold_6 | 65899215 | 65899457 |
| scaffold_2 | 90082675 | 90082850 |
| scaffold_2 | 45648573 | 45648704 |
| scaffold_10 | 46239592 | 46239793 |
| scaffold_31 | 17811929 | 17812041 |
| scaffold_22 | 3209376 | 3209491 |
| scaffold_33 | 2624404 | 2624623 |
| scaffold_0 | 220810084 | 220810236 |
| scaffold_45 | 4457534 | 4457636 |
| scaffold_0 | 19094594 | 19094694 |
| scaffold_7 | 15516433 | 15516528 |
| scaffold_3 | 38128986 | 38129133 |
| scaffold_12 | 28364487 | 28364704 |
| scaffold_34 | 4301026 | 4301187 |
| scaffold_0 | 187893529 | 187893812 |
| scaffold_5 | 8164287 | 8164373 |
| scaffold_1 | 102361970 | 102362116 |
| scaffold_3 | 6951744 | 6951853 |
| scaffold_2 | 13527644 | 13528097 |
| scaffold_8 | 35652365 | 35652442 |
| scaffold_3 | 27019769 | 27019916 |
| scaffold_35 | 281510 | 281564 |
| scaffold_29 | 26268335 | 26268472 |
| scaffold_13 | 41328125 | 41328356 |
| scaffold_61 | 1889662 | 1889727 |
| scaffold_0 | 147163257 | 147163431 |
| scaffold_32 | 10370015 | 10370095 |
| scaffold_2 | 23744711 | 23744782 |

### Example 2

Adaptation of CHO Cells with Media Supplements

### Wet-Lab Methodology

For this experiment, a transgenic CHO cell line, Agarabi CHO (ATCCR CRL-3440™), was adapted for 2 weeks in CD FortiCHO medium supplemented with 8 mM L-glutamine & 1 mg/L human insulin-like growth factor 1 (IGF-1) at 37° C., 8% CO2, at a shaking speed of 130 RPM. Batch culture of 6 flasks was maintained for 7 days where 3 flasks represent technical replicates for the control set (without IGF-1 adaptation) and 3 flasks represent technical replicates for the IGF-1 adapted set. The flasks were seeded with 3E5 viable cells/mL on day 0 and 1 mg/L Insulin Growth Factor was added to the adapted set. Cell count, cell viability, and protein production were measured every 2 days and cell pellets were collected for both control and treatment set on day 7. Adaptation of CHO cells with IGF-1 had no significant effect on growth rate and viability, however, heterologous protein productivity was doubled as compared to the control set.

Genomic DNA was purified from the collected cell pellets using DNeasy Blood & Tissue Kit (Qiagen) and was quantified using PicroGreen or NanoDrop™ 2000. The genomic DNA (500 ng) from the control and adapted set were used to prepare libraries for Whole Genome Bisulfite Sequencing (WGBS). The sequencing of the libraries was performed by a third party on a NovaSeq platform which generated 125 GB of data per sample.

### Computational Methodology

Raw sequencing data were conducted quality control (fastqc) 1, sequencing adaptors trimming (TrimGalore) 2, and alignment with Bismark3. CMV promoter combined with CHOK1-GS (Cricetulus griseus) genome was used as a reference genome.

Bismark was also used for removing duplicated reads and extracting methylation counts from alignment output. SNPs were filtered out, and only counts with a minimum coverage of 10× were used for the downstream analysis, which resulted in 4244091 CpG sites for IGF-1 adapted samples.

Since regulated methylation targets are most commonly clustered into short regions, DMRfinder4 was used to perform a modified single-linkage clustering of methylation sites. With a maximum distance between CpG sites of 100 bp, 2048904 genomic regions were found for IGF-1 adapted samples.

### Differential Methylation Analysis

Differential methylation analysis was performed using MethylKit5 between the control and adapted groups. Logistic regression was used to determine the differential methylation across all regions, and the sliding linear model (SLIM) 6 method to do FDR correction. Regions with FDR corrected p-value <0.05 and methylation change greater than 25% between groups were determined as differentially methylated regions (DMRs), which was 289 for IGF-1 adapted samples listed in Table 2. Principal Component Analysis (PCA) is a dimensionality reduction technique that emphasizes variation in a dataset. PCA analysis for DMRs is shown in FIG. 2.

Preliminary results show DMRs play roles in epigenetic changes of IGF-1 adaptation, which can be potentially used as markers for future research.

### Example 3

### Wet-Lab Methodology

### Media Adaptation of Humira Cells

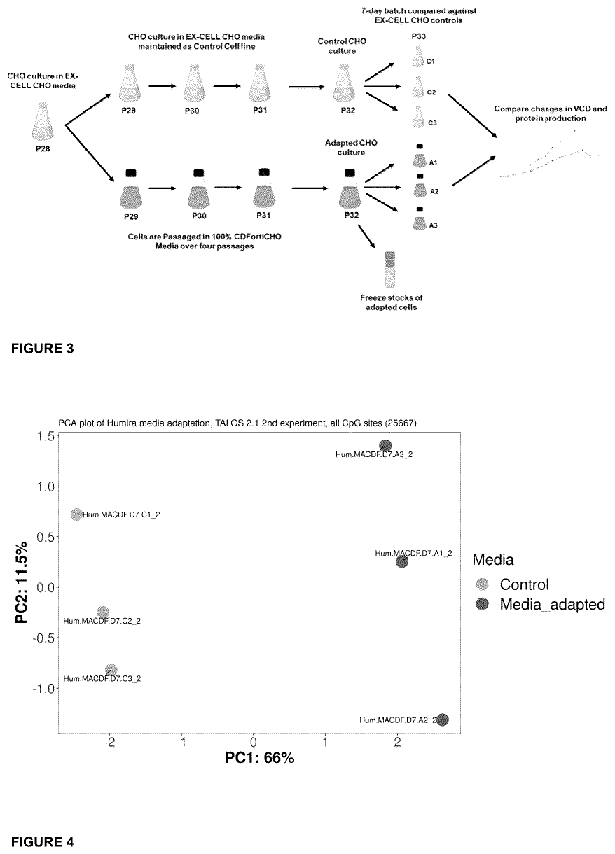

Humira431 cells (A*STAR Bioprocessing Technology Institute) were initially grown in EX-CELL Advanced CHO medium (Sigma-Aldrich, 14366C). At passage 28 (P28), Humira431 cells were transferred to and adapted in the new media, CDFortiCHO (ThermoFisher) for 4 passages over 2 weeks while control Humira431 cells were continuously grown in EX-CELL Advanced CHO medium. Adapted and control Humira431 cells at passage 32 (P32) were split into 3 flasks each to obtain biological replicates and cultured for 7 days. Viable cell density (VCD) was measured across 7 days. At day 7, media and cell pellets were collected from both adapted and control flasks for Cedex analysis and genomic DNA (gDNA) isolation (FIG. 3).

### DNA Extraction

DNA is extracted using the PureLink Genomic DNA Isolation Minikit kit (Invitrogen), including RNAase treatment following the manufacturer's instructions. DNA quantity is measured by PicoGreen assay and DNA quality is assessed via NanoDrop (Thermo Scientific) to ensure the A260/280 ratio is ≤1.8. A small amount of sample is then also analysed on an agarose gel to ensure each sample contains high molecular weight DNA.

### Bisulfite Conversion and BeadChip Analysis

The genomic DNA samples were then subjected to bisulfite conversion using the EZ DNA Methylation-Gold™ Kit (Zymo Research). The methylation levels were then quantified using our customized methylation BeadChip kits (Illumina) which can analyze over 50,000 methylation sites quantitatively across the genome at single-nucleotide resolution.

### Data Processing

The customized chip array data processing was performed in R version 4.1.2 using sesame version 1.14.2. DNA methylation level for each site was calculated as methylation β-value. Beta values are defined as methylated signal/(methylated signal+unmethylated signal). It can be computed using getBetas function. The SeSAMe pipeline (Zhou et al. 2018) was used to generate normalized β-values and for quality control. Low intensity-based detection calling and making (based on p-value) were done with pOOBAH. Background subtraction based on normal-exponential deconvolution using out-of-band probes noob (Triche et al. 2013) and optionally with extra bleed-through subtraction were also implemented.

### Results

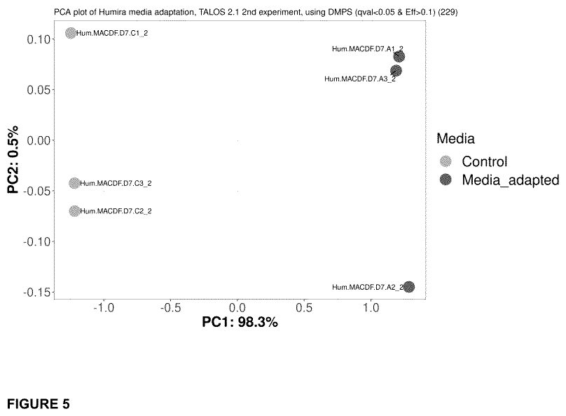

Plotting of the first two principal components of the CpGs before and after differential methylation analysis reveal meaningful clustering of the samples. Differentially methylated Positions (DMPs) were able to effectively cluster of the CHO samples by the media adaptation from the control samples. List of DMPs identified is shown in Table 3. FIG. 4 and FIG. 5 show the PCA analysis using all methylated CpG sites and differentially methylated sites respectively.

TABLE 2a
List of differentially methylated regions (DMRs)
identified in IGF-1 adapted samples.

| chr | start | end |
|---|---|---|
| scaffold_8 | 14408898 | 14409225 |
| scaffold_2 | 50421541 | 50421665 |
| scaffold_22 | 29532421 | 29532895 |
| scaffold_38 | 2269741 | 2270047 |
| scaffold_21 | 29866209 | 29866421 |
| scaffold_54 | 2515721 | 2515764 |
| scaffold_0 | 25949303 | 25949594 |
| scaffold_35 | 13205057 | 13205465 |
| scaffold_37 | 12066215 | 12066671 |
| scaffold_22 | 12939539 | 12939845 |
| scaffold_44 | 8069279 | 8069312 |
| scaffold_6 | 60778300 | 60778324 |
| scaffold_19 | 18239123 | 18239497 |
| scaffold_0 | 18663494 | 18663945 |
| scaffold_10 | 11412124 | 11412475 |
| scaffold_9 | 71462 | 71763 |
| scaffold_1 | 112831481 | 112831557 |
| scaffold_26 | 7687221 | 7687397 |
| scaffold_7 | 65770953 | 65771379 |
| scaffold_3 | 118117169 | 118117489 |
| scaffold_2 | 6797731 | 6797826 |
| scaffold_0 | 134717628 | 134718015 |
| scaffold_12 | 53824934 | 53825407 |
| scaffold_1 | 37231683 | 37231892 |
| scaffold_4 | 36243642 | 36243805 |
| scaffold_7 | 62301087 | 62301555 |
| scaffold_29 | 22627871 | 22628150 |
| scaffold_1 | 149299846 | 149300033 |
| scaffold_67 | 1955703 | 1955843 |
| scaffold_1 | 130672847 | 130673013 |
| scaffold_1 | 40142460 | 40142639 |
| scaffold_3 | 63860745 | 63861232 |
| scaffold_21 | 10588291 | 10588338 |
| scaffold_12 | 36695387 | 36695839 |
| scaffold_3 | 78726601 | 78726909 |
| scaffold_7 | 38240253 | 38240638 |
| scaffold_3 | 115750196 | 115750260 |
| scaffold_1 | 147219793 | 147220137 |
| scaffold_19 | 2338298 | 2338596 |
| scaffold_1 | 161713879 | 161713984 |
| scaffold_39 | 4602534 | 4602598 |
| scaffold_14 | 11750971 | 11751304 |
| scaffold_6 | 66885025 | 66885493 |
| scaffold_2 | 8247361 | 8247759 |
| scaffold_0 | 177213470 | 177213608 |
| scaffold_39 | 5248125 | 5248496 |
| scaffold_9 | 24985783 | 24986074 |
| scaffold_6 | 72646272 | 72646537 |
| scaffold_6 | 16347716 | 16347802 |
| scaffold_110 | 113119 | 113421 |
| scaffold_27 | 5171198 | 5171687 |
| scaffold_9 | 6180957 | 6181129 |
| scaffold_10 | 14215150 | 14215392 |
| scaffold_20 | 21187571 | 21187927 |
| scaffold_19 | 34385730 | 34385826 |
| scaffold_3 | 97436277 | 97436398 |
| scaffold_35 | 13446725 | 13446964 |
| scaffold_36 | 15105267 | 15105429 |
| scaffold_9 | 9490960 | 9491438 |
| scaffold_0 | 10930489 | 10930570 |
| scaffold_9 | 29181906 | 29182190 |
| scaffold_3 | 20007124 | 20007431 |
| scaffold_12 | 48007028 | 48007336 |
| scaffold_51 | 745700 | 746028 |
| scaffold_12 | 52908876 | 52909134 |
| scaffold_57 | 1909218 | 1909382 |
| scaffold_0 | 211532586 | 211532636 |
| scaffold_15 | 12787266 | 12787682 |
| scaffold_22 | 12856162 | 12856490 |
| scaffold_5 | 56486953 | 56487265 |
| scaffold_8 | 17571055 | 17571111 |
| scaffold_8 | 38858091 | 38858336 |
| scaffold_30 | 19817051 | 19817193 |
| scaffold_44 | 375690 | 375913 |
| scaffold_24 | 1318846 | 1319229 |
| scaffold_7 | 66072324 | 66072517 |
| scaffold_0 | 221591372 | 221591408 |
| scaffold_10 | 58195034 | 58195135 |
| scaffold_8 | 71897332 | 71897563 |
| scaffold_0 | 161268656 | 161268801 |
| scaffold_0 | 46663770 | 46663909 |
| scaffold_4 | 5608305 | 5608411 |
| scaffold_29 | 23120677 | 23120919 |
| scaffold_42 | 7419267 | 7419350 |
| scaffold_6 | 50789763 | 50789821 |
| scaffold_1 | 40394347 | 40394539 |
| scaffold_4 | 4160072 | 4160309 |
| scaffold_19 | 20192868 | 20193186 |
| scaffold_6 | 63726426 | 63726528 |
| scaffold_5 | 5246222 | 5246424 |
| scaffold_13 | 33985232 | 33985382 |
| scaffold_20 | 10454055 | 10454187 |
| scaffold_0 | 36079659 | 36079667 |
| scaffold_0 | 26801285 | 26801377 |
| scaffold_34 | 14457203 | 14457559 |
| scaffold_30 | 1812957 | 1812983 |
| scaffold_43 | 1484770 | 1485027 |
| scaffold_1 | 132260978 | 132261193 |
| scaffold_12 | 54208084 | 54208161 |
| scaffold_6 | 58076177 | 58076403 |
| scaffold_7 | 17882389 | 17882563 |
| scaffold_35 | 14679336 | 14679438 |
| scaffold_33 | 7310947 | 7311068 |
| scaffold_22 | 32895517 | 32895962 |
| scaffold_2 | 46646070 | 46646163 |
| scaffold_9 | 53765494 | 53765684 |
| scaffold_26 | 7603316 | 7603392 |
| scaffold_15 | 24566050 | 24566235 |
| scaffold_30 | 19817051 | 19817193 |
| scaffold_44 | 375690 | 375913 |
| scaffold_24 | 1318846 | 1319229 |
| scaffold_7 | 66072324 | 66072517 |
| scaffold_0 | 221591372 | 221591408 |
| scaffold_10 | 58195034 | 58195135 |
| scaffold_8 | 71897332 | 71897563 |
| scaffold_0 | 161268656 | 161268801 |
| scaffold_0 | 46663770 | 46663909 |
| scaffold_4 | 5608305 | 5608411 |
| scaffold_29 | 23120677 | 23120919 |
| scaffold_42 | 7419267 | 7419350 |
| scaffold_6 | 50789763 | 50789821 |
| scaffold_1 | 40394347 | 40394539 |
| scaffold_4 | 4160072 | 4160309 |
| scaffold_19 | 20192868 | 20193186 |
| scaffold_6 | 63726426 | 63726528 |
| scaffold_5 | 5246222 | 5246424 |
| scaffold_13 | 33985232 | 33985382 |
| scaffold_20 | 10454055 | 10454187 |
| scaffold_0 | 36079659 | 36079667 |
| scaffold_0 | 26801285 | 26801377 |
| scaffold_34 | 14457203 | 14457559 |
| scaffold_30 | 1812957 | 1812983 |
| scaffold_43 | 1484770 | 1485027 |
| scaffold_1 | 132260978 | 132261193 |
| scaffold_12 | 54208084 | 54208161 |
| scaffold_6 | 58076177 | 58076403 |
| scaffold_7 | 17882389 | 17882563 |
| scaffold_35 | 14679336 | 14679438 |
| scaffold_33 | 7310947 | 7311068 |
| scaffold_22 | 32895517 | 32895962 |
| scaffold_2 | 46646070 | 46646163 |
| scaffold_9 | 53765494 | 53765684 |
| scaffold_26 | 7603316 | 7603392 |
| scaffold_15 | 24566050 | 24566235 |

TABLE 2b
List of differentially methylated regions (DMRs)
identified in IGF-1 adapted samples.

| chr | start | end |
|---|---|---|
| scaffold_4 | 46838477 | 46838525 |
| scaffold_1 | 40452365 | 40452454 |
| scaffold_4 | 31231044 | 31231111 |
| scaffold_59 | 2358940 | 2359226 |
| scaffold_2 | 40270729 | 40271062 |
| scaffold_21 | 33819495 | 33819720 |
| scaffold_36 | 554210 | 554243 |
| scaffold_0 | 24828369 | 24828530 |
| scaffold_0 | 84626749 | 84626904 |
| scaffold_15 | 23110977 | 23111032 |
| scaffold_51 | 1029523 | 1029755 |
| scaffold_54 | 2943156 | 2943282 |
| scaffold_30 | 12227834 | 12227959 |
| scaffold_20 | 5206974 | 5207142 |
| scaffold_8 | 27498735 | 27498750 |
| scaffold_6 | 23334205 | 23334252 |
| scaffold_21 | 115674 | 115770 |
| scaffold_5 | 71484400 | 71484510 |
| scaffold_5 | 76487736 | 76487905 |
| scaffold_294 | 30527 | 30664 |
| scaffold_18 | 362609 | 362731 |
| scaffold_39 | 5115921 | 5115984 |
| scaffold_0 | 145623209 | 145623306 |
| scaffold_17 | 37526021 | 37526026 |
| scaffold_5 | 62406675 | 62406812 |
| scaffold_10 | 40092253 | 40092370 |
| scaffold_12 | 34496664 | 34496798 |
| scaffold_17 | 37823878 | 37823900 |
| scaffold_177 | 108918 | 109057 |
| scaffold_43 | 2789548 | 2789652 |
| scaffold_5891 | 625 | 1001 |
| scaffold_1 | 15992497 | 15992536 |
| scaffold_12 | 19536793 | 19536897 |
| scaffold_44 | 2759370 | 2759457 |
| scaffold_0 | 208515585 | 208515621 |
| scaffold_0 | 44169368 | 44169524 |
| scaffold_0 | 127708478 | 127708500 |
| scaffold_12 | 28190332 | 28190769 |
| scaffold_4 | 39792499 | 39792654 |
| scaffold_5 | 3862876 | 3863105 |
| scaffold_4 | 82124551 | 82124691 |
| scaffold_15 | 10894110 | 10894291 |
| scaffold_67 | 566792 | 566824 |
| scaffold_2 | 13520901 | 13521043 |
| scaffold_4 | 38044709 | 38044879 |
| scaffold_24 | 26202059 | 26202329 |
| scaffold_27 | 875976 | 876003 |
| scaffold_45 | 2128856 | 2128976 |
| scaffold_37 | 2439671 | 2439833 |
| scaffold_21 | 21559867 | 21559952 |
| scaffold_8 | 65007115 | 65007319 |
| scaffold_8 | 53964131 | 53964301 |
| scaffold_3 | 58320469 | 58320666 |
| scaffold_16 | 20587686 | 20587832 |
| scaffold_43 | 5979577 | 5979789 |
| scaffold_14 | 35705378 | 35705569 |
| scaffold_17 | 9641455 | 9641523 |
| scaffold_6015 | 205 | 285 |
| scaffold_2 | 4637629 | 4637789 |
| scaffold_9 | 15607400 | 15607517 |
| scaffold_12 | 47812083 | 47812228 |
| scaffold_3 | 24837697 | 24837987 |
| scaffold_8 | 67097612 | 67097829 |
| scaffold_7 | 12194341 | 12194440 |
| scaffold_27 | 3759367 | 3759442 |
| scaffold_30 | 6458299 | 6458317 |
| scaffold_4 | 17114849 | 17114949 |
| scaffold_24 | 15714804 | 15714866 |
| scaffold_16 | 39261687 | 39261846 |
| scaffold_29 | 14743908 | 14743981 |
| scaffold_19 | 5923042 | 5923155 |
| scaffold_24 | 22880118 | 22880129 |
| scaffold_10 | 1691212 | 1691335 |
| scaffold_5 | 24215603 | 24215652 |
| scaffold_3 | 1539960 | 1539970 |
| scaffold_22 | 7204758 | 7204837 |
| scaffold_6 | 69128866 | 69129097 |
| scaffold_5 | 56754022 | 56754033 |
| scaffold_2 | 25801677 | 25801855 |
| scaffold_2760 | 2736 | 2905 |
| scaffold_30 | 9841394 | 9841503 |
| scaffold_13 | 14205334 | 14205434 |
| scaffold_62 | 366037 | 366198 |
| scaffold_9 | 63865309 | 63865381 |
| scaffold_21 | 33052401 | 33052490 |
| scaffold_12 | 18894036 | 18894186 |
| scaffold_0 | 4927850 | 4927991 |
| scaffold_1 | 112272095 | 112272230 |
| scaffold_16 | 11622901 | 11623001 |
| scaffold_12 | 24914255 | 24914481 |
| scaffold_2 | 3617470 | 3617546 |
| scaffold_0 | 128210855 | 128211154 |
| scaffold_27 | 16149908 | 16150010 |
| scaffold_0 | 207596754 | 207596781 |
| scaffold_12 | 42555855 | 42556032 |
| scaffold_0 | 36074997 | 36075058 |
| scaffold_16 | 20587336 | 20587379 |
| scaffold_41 | 8837831 | 8837936 |
| scaffold_21 | 32448146 | 32448250 |
| scaffold_2589 | 1120 | 1221 |
| scaffold_1 | 53918654 | 53918783 |
| scaffold_5 | 77039891 | 77039983 |
| scaffold_5 | 57947700 | 57947908 |
| scaffold_3 | 73955480 | 73955543 |
| scaffold_20 | 29518333 | 29518425 |
| scaffold_777 | 4190 | 4244 |
| scaffold_29 | 23616788 | 23616848 |
| scaffold_46 | 1615662 | 1615869 |
| scaffold_10 | 1691212 | 1691335 |
| scaffold_5 | 24215603 | 24215652 |
| scaffold_3 | 1539960 | 1539970 |
| scaffold_22 | 7204758 | 7204837 |
| scaffold_6 | 69128866 | 69129097 |
| scaffold_5 | 56754022 | 56754033 |
| scaffold_2 | 25801677 | 25801855 |
| scaffold_2760 | 2736 | 2905 |
| scaffold_30 | 9841394 | 9841503 |
| scaffold_13 | 14205334 | 14205434 |
| scaffold_62 | 366037 | 366198 |
| scaffold_9 | 63865309 | 63865381 |
| scaffold_21 | 33052401 | 33052490 |
| scaffold_12 | 18894036 | 18894186 |
| scaffold_0 | 4927850 | 4927991 |
| scaffold_1 | 112272095 | 112272230 |
| scaffold_16 | 11622901 | 11623001 |
| scaffold_12 | 24914255 | 24914481 |
| scaffold_2 | 3617470 | 3617546 |
| scaffold_0 | 128210855 | 128211154 |
| scaffold_27 | 16149908 | 16150010 |
| scaffold_0 | 207596754 | 207596781 |
| scaffold_12 | 42555855 | 42556032 |
| scaffold_0 | 36074997 | 36075058 |
| scaffold_16 | 20587336 | 20587379 |
| scaffold_41 | 8837831 | 8837936 |
| scaffold_21 | 32448146 | 32448250 |
| scaffold_2589 | 1120 | 1221 |
| scaffold_1 | 53918654 | 53918783 |
| scaffold_5 | 77039891 | 77039983 |
| scaffold_5 | 57947700 | 57947908 |
| scaffold_3 | 73955480 | 73955543 |
| scaffold_20 | 29518333 | 29518425 |
| scaffold_777 | 4190 | 4244 |
| scaffold_29 | 23616788 | 23616848 |
| scaffold_46 | 1615662 | 1615869 |

TABLE 3
Differentially methylated probes (CpG sites) identified between control and adapted samples

| | | | | |
|---|---|---|---|---|
| cg112283583_TC21 | cg118360319_TC21 | cg120333741_BC21 | cg117194987_BC21 | cg116430870_TC21 |
| cg118805358_BC21 | cg116815581_BC21 | cg122182443_BO21 | cg116087494_BC21 | cg112292284_TC21 |
| cg117114250_TC21 | cg115341085_BC21 | cg120472251_TO21 | cg109549903_BC21 | cg113157700_BC21 |
| cg111580700_TC21 | cg120568539_TC21 | cg109557619_BC21 | cg116391883_TC21 | cg115935274_TC21 |
| cg121042670_TC21 | cg111189892_BC21 | cg115932506_BC21 | cg109505181_BC21 | cg116055476_BC21 |
| cg122459456_BC21 | cg111636260_BC21 | cg116224332_TC21 | cg118466413_TC21 | cg111639846_TC21 |
| cg118804598_TC21 | cg115041267_TO11 | cg122603899_BC21 | cg112284811_BC21 | cg115872482_BC21 |
| cg117643829_BO21 | cg118311809_BC21 | cg114608117_BC21 | cg110188241_TC21 | cg164834675_TC21 |
| cg116387747_BC21 | cg118311754_BC21 | cg115773153_BC21 | cg109589376_TC21 | cg115294976_BO11 |
| cg114223346_TC21 | cg121054680_BC21 | cg109518525_BC21 | cg119415987_BC21 | cg111679376_TC21 |
| cg109413061_BC21 | cg118466416_TC21 | cg115264077_BC21 | cg114680485_TC21 | cg122566360_TC21 |
| cg110604689_BC21 | cg110675955_TC21 | cg115773154_TC21 | cg119263528_TC21 | cg109413045_BC21 |
| cg120521153_BC21 | cg122434585_TC21 | cg111301874_BC21 | cg110319834_TC21 | cg121662924_BC21 |
| cg119487093_TC21 | cg110608130_BC21 | cg117931636_TC21 | cg119547223_BC21 | cg110259919_BC21 |
| cg120398192_TC21 | cg163986267_BC21 | cg113715109_TC21 | cg123137660_BC21 | cg119764866_BC21 |
| cg116832993_BC21 | cg121222977_BO21 | cg121115608_TC21 | cg118010113_TC21 | cg118684128_TC21 |
| cg120936709_BC21 | cg114817607_BC21 | cg111454550_TC21 | cg114481809_BC21 | cg111293214_TC21 |
| cg120901331_BC21 | cg115773151_TC21 | cg123037805_BC21 | cg165229998_BC21 | cg119303585_TC21 |
| cg121787882_TC21 | cg113597101_BC21 | cg120233150_BC21 | cg109872303_BC21 | cg113357565_TC21 |
| cg116660736_TC21 | cg111356972_BC21 | cg115872317_BC21 | cg117340478_BO11 | cg117114248_TC21 |
| cg109873239_BC21 | cg115889304_TC21 | cg119136806_TC21 | cg112283373_TC21 | cg117114246_TC21 |
| cg122351655_BC21 | cg119408378_BC21 | cg113715203_BC21 | cg114680354_TC21 | cg112725246_TC21 |
| cg117136418_BC21 | cg113716445_BC21 | cg110330926_TC21 | cg114282048_BC21 | cg119725687_BC21 |
| cg113714871_TC21 | cg110797346_BC21 | cg116224351_TC21 | cg114386020_BC21 | cg112120215_BC21 |
| cg113276827_TC21 | cg116808930_TC21 | cg112284812_BC21 | cg123242353_TC21 | cg115428793_BC21 |
| cg118627620_BC21 | cg111924859_TC21 | cg111909606_BO21 | cg118720010_BC21 | cg122601060_BC21 |
| cg109613595_TC21 | cg117180901_BC21 | cg123562223_BC21 | cg110696365_BC21 | cg117195515_TC21 |
| cg120845207_BC21 | cg118238087_TC21 | cg114282047_BO11 | cg111142991_BC21 | cg122566342_BC21 |
| cg111521090_BC21 | cg119524339_BC21 | cg121587608_BC21 | cg112181813_TC21 | cg116719650_BC21 |
| cg114371858_BC21 | cg122697821_BC21 | cg121245789_TC21 | cg111757581_BC21 | cg123340186_TC21 |
| cg121114450_TC21 | cg122697822_BO11 | cg114230872_BC21 | cg115492533_TC21 | cg109838988_BC21 |
| cg123249953_TC21 | cg113664074_TC21 | cg119297609_BC21 | cg113172673_BC21 | cg113996752_BC21 |
| cg114371860_TC21 | cg115773152_BC21 | cg119144439_TC21 | cg116391860_TC21 | cg117528162_TC21 |
| cg110188226_TC21 | cg120826114_TC21 | cg114228690_BC21 | cg110319331_BC21 | cg112188603_BC21 |
| cg165539646_BC21 | cg116058772_BC21 | cg114115243_TO21 | cg110196760_TC21 | cg115473762_TC21 |
| cg109498205_BC21 | cg117617616_BO21 | cg115848521_TO21 | cg120567395_TC21 | cg123562222_TC21 |
| cg112502952_BC21 | cg111842598_TC21 | cg120567390_TC21 | cg113715191_BC21 | cg113949955_TC21 |
| cg120618202_BC21 | cg114941096_BC21 | cg111635676_BC21 | cg122941293_TC21 | cg119377506_BC21 |
| cg115044603_BC21 | cg114791808_TC21 | cg113715702_BC21 | cg122866214_BC21 | cg117869932_BC21 |
| cg117133844_BO11 | cg109644940_TC21 | cg113573643_BC21 | cg114286985_TC11 | cg114910783_TC21 |
| cg115044602_BC21 | cg112181822_TC21 | cg116520214_BO21 | cg114626281_BC21 | cg122186988_BC21 |
| cg114193015_BC21 | cg115323635_TC21 | cg121104125_TC21 | cg112813038_BC21 | cg123621425_BC21 |
| cg115564972_TC21 | cg111842557_TC21 | cg111366955_BC21 | cg120609662_BC21 | cg118360330_BC21 |
| cg111304610_BC21 | cg122124885_TC21 | cg117645303_BC21 | cg118311808_BO21 | cg109782559_TC21 |
| cg117136417_BC21 | cg120499893_BC21 | cg117128805_BC21 | cg119724201_TC21 | cg121511527_BO11 |
| cg119391909_TC21 | cg115567814_TC21 | cg118278735_BC21 | cg112437567_BC21 | |

1. A DNA array-based method of assessing the effect of at least one test component of cell medium on at least one phenotype of interest of a test mammalian cell line cultured in cell media comprising the test component, the method comprising the steps of:
(a) determining a test methylation profile of one or more pre-selected methylation sites within the DNA of the test cell line;
(b) comparing the test methylation profile obtained from (a) with at least one control methylation profile from the same strain of mammalian cell line cultured in cell media without the test component; and
wherein a significant similarity in the test methylation profile of (a) compared to the control methylation profile, is indicative of the test cell having the phenotype of interest and the test component not having an effect on the phenotype of interest; and
wherein a significant difference in the test methylation profile of (a) compared to the control methylation profile, is indicative of the test cell having the phenotype of interest and the test component having an effect on the phenotype of interest and wherein the method comprises a further step of:
(c) comparing the test methylation profile obtained from (a) with
(i) at least one first reference methylation profile obtained from a first mammalian reference cell line that displays at least one phenotype of interest; and/or
(ii) at least one second reference methylation profile obtained from a second mammalian reference cell line that does not display the phenotype of interest; and
wherein the reference cell lines are not in contact with the test component; and
wherein a significant similarity in the test methylation profile of (a) compared to the first or second reference methylation profile, is indicative of the test cell having the phenotype of interest or not having the phenotype of interest respectively; and
wherein a difference in the test methylation profile of (a) compared to the first or second reference methylation profile, is indicative of the test cell not having the phenotype of interest or having the phenotype of interest.

2. The method according to claim 1, wherein the pre-selected methylation sites is related to at least one phenotype of interest in the test cell line.

3. The method according to either claim 1, wherein the phenotype of interest is selected from the group consisting of phenotypic homogeneity, protein quality, optimal carbohydrate metabolism, optimal amino acid metabolism, optimal lipid metabolism, optimal heterologous protein production, optimal cell survivability and combinations thereof.

4. The method according to claim 1, wherein the first reference methylation profile is a compilation of more than one CpG site from at least one reference cell line that displays at least one phenotype of interest; and the second reference methylation profile is a compilation of more than one CpG site from at least one reference cell line that does not display at least one phenotype of interest.

5. The method according to claim 1, wherein the component of the cell media is selected from the group consisting of amino acids, small peptides, buffering agents, a carbohydrate, inorganic salts, serum or parts thereof, vitamins and minerals.

6. The method according to claim 1, wherein the mammalian cell line is from a mammal selected from the group consisting of a mouse, a rat, a guinea pig, a dog, a mini-pig, a human being, a cow, a sheep, a pig, a goat, a horse, a donkey, a mule, and a hamster.

7. The method according to claim 1, wherein the mammalian cell is an immortalized cell line.

8. The method according to claim 7, wherein the immortalized cell line is selected from the group consisting of CHO, BHK, Vero, HEK293, HEK 293T, HeLa cell, NS0 cell, Sp2/0 cell, and derivatives thereof.

9. A DNA array-based method of assessing the effect of at least one test component of cell media on the production of at least one biosimilar from an immortalised test cell line,
wherein the biosimilar is significantly similar relative to an innovator protein produced by an immortalized reference cell line which is the same cell line as the test cell line, the method comprising the steps of:
(a) determining a first test methylation profile from DNA obtained from the immortalized test cell line that is cultured in the cell media comprising the test component, and
(b) determining a second methylation profile from DNA obtained from the immortalized test cell line that is cultured in the test component absent cell media;
(c) comparing the test methylation profile obtained from (a) and (b) with a reference methylation profile obtained from a immortalized reference cell line;
wherein a significant similarity between the test methylation profile of (a) and the reference methylation profile and a difference between the test methylation profile of (b) and the reference methylation profile is indicative of the two cell lines producing biosimilars and the test component having a positive effect on the production of biosimilars from immortalized cell lines.

10. A DNA array-based method of assessing the effect of at least one test component of cell media on the production of at least one bioidentical from an immortalised test cell line,
wherein the bioidentical is significantly similar relative to an innovator protein produced by an immortalized reference cell line which is the same cell line as the test cell line, the method comprising the steps of:
(a) determining a first test methylation profile from DNA obtained from the immortalized test cell line that is cultured in the cell media comprising the test component, and
(b) determining a second methylation profile from DNA obtained from the immortalized test cell line that is cultured in the test component absent cell media;
(c) comparing the test methylation profile obtained from (a) and (b) with a reference methylation profile obtained from a immortalized reference cell line;
wherein a significant similarity between the test methylation profile of (a) and the reference methylation profile and a difference between the test methylation profile of (b) and the reference methylation profile is indicative of the two cell lines producing bioidenticals and the test component having a positive effect on the production of bioidenticals from immortalized cell lines.

11. The method according to claim 1, wherein the DNA methylation-based array is a bead-based array.

12. Use of a DNA-methylation based array for determining the effect of at least one test component of cell media on producing mammalian cell lines displaying at least one phenotype of interest.

13. DNA methylation-based array for determining the effect of at least one test component of cell media on producing mammalian cell lines displaying at least one phenotype of interest.
